(19) United States
(12) Patent Application Publication
(10) Pub. No.: US 2023/0240941 A1
(43) Pub. Date: Aug. 3, 2023

(54) APPARATUS AND METHOD FOR STERILIZING AND ASSEMBLING A CARTRIDGE CONTAINING A MEDICAL SUBSTANCE TO A DEVICE FOR DISPENSING SAID MEDICAL SUBSTANCE

(71) Applicant: Stevanato Group S.p.A., Piombino Dese (IT)

(72) Inventors: Irio Calasso, Piombino Dese (IT); Matteo De Donatis, Piombino Dese (IT); Nadine Troes, Piombino Dese (IT)

(73) Assignee: Stevanato Group S.p.A., Piombino Dese (IT)

(21) Appl. No.: 18/009,261

(22) PCT Filed: Jun. 9, 2021
(86) PCT No.: PCT/IB2021/055066

§ 371 Date: Dec. 8, 2022

(30) Foreign Application Priority Data
Jun. 12, 2020 (IT) 102020000014101

(51) Int. Cl. A61J 1/14; A61L 2/08

(52) U.S. Cl. CPC A61J 1/1468; A61L 2/08

(57) ABSTRACT

An apparatus (4) for sterilizing and assembling a cartridge (8) containing a medical substance to a device (12) for dispensing said medical substance, comprising: a cartridge (8) containing the medical substance to be dispensed, the cartridge being provided with a cap (24), a device (12) for dispensing said medical substance, the device being provided with at least one needle (28) configured to pierce the cap (24) of the cartridge (8) and allow the withdrawal of the medical substance from the cartridge (8), a first interface (32) that may be sealingly joined to the cap (24) of the cartridge (8) by means of the interposition of a first gasket (36), a second interface (40) joined to the dispensing device (12) near said needle (28), wherein the second interface (40) has a seal (44) that isolates the needle (28) from the external environment in a sterile volume (48), and is provided with a second gasket (52) which may be sealingly joined to the first interface (32) in order to identify a sealed chamber (56) delimited between a portion of the cap (24) facing said needle (28) and said seal (44), the second gasket (52) being external to said sterile volume (48), and a sterilizing device (80) configured to irradiate and sterilize said sealed chamber (56).

### FIELD OF APPLICATION

This invention relates to an apparatus and a method for sterilizing and assembling a cartridge containing a medical substance to a device for dispensing said medical substance.

### PRIOR ART

As is known, some medical conditions require a regular dose of a continuous infusion of medicines, while others require punctual or periodic administration. These medicines are often provided as liquid solutions to be infused, for example transdermally, intramuscularly or intravenously. Diabetic patients, for example, may require insulin. In an effort to make the lives of these patients easier, various infusion devices have been developed.

The infusion devices known in the prior art typically comprise simple injection pen devices or complex pump devices, which use a mechanical or electromechanical pump to deliver the medicine to a patient through the skin.

In particular, some pump devices are part of an administration system which comprises the administration or dispensing device applied in direct contact with the skin of the patient (also known as a POD), and these devices may function autonomously or may also have a (handheld) device for activating or controlling the administration or dispensing device. Some other pump solutions have an administration device connected via tubes to a needle which infuses the medicine into the patient. The POD administration device is fixedly applied to the skin, usually by means of an adhesive, until the medical liquid housed in a suitable reservoir joined to the device has been completely depleted or until after a specified time interval. The dispensing device may also comprise a number of parts that are coupled to one another before use, for example a disposable part and a part that is reused. The dispensing device may also comprise buttons for activating and/or for regulating the dose. Moreover, the device may also comprise a display or LED lights or a sound emitter in order to inform the user of the operating state of the dispensing device.

In another solution, owned by the proprietor of the present patent application, the activation device is instead a portable device which, when required, is picked up by the user and brought into proximity with the administration device (a few millimeters away). The activation device contains magnets which, when applied at close range to the administration device, activate the relevant administration means. In particular, the administration device comprises a rotor which rotates by means of magnetic coupling and is operatively connected to a pump which is in turn connected, at the inlet, to the reservoir for the medical substance to be dispensed and, at the outlet, to a dispensing needle which penetrates the skin of the patient.

One critical element of the system is the fact that manufacturers of the medical substance dispensing device, in general, do not normally also produce the medical substance that is normally contained in suitable container vials of various geometries and materials. Pharmaceutical manufacturers therefore produce vials containing the medical substance to be dispensed that have to be suitably inserted or assembled inside the dispensing device. Alternatively, the medical contents are transferred by means of a syringe or suitable apparatus into a container inside the dispenser. Of course, when the vial is inserted into the dispenser, the assembly step has to guarantee the sterility of the medical substance and of all the fluid part of the dispenser that is to be dispensed later.

Vials produced by pharmaceutical manufacturers are filled in an aseptic environment and, after filling, are at least partially sterile but, in the step of assembly to the dispensing device, which normally takes place in a non-sterile environment, this sterility is inevitably lost, unless a series of measures are undertaken that considerably increase the time and the costs for assembling the cartridge to the dispensing device.

In the known solutions, this assembly normally takes place in a sterile environment or chamber, after both the cartridge and the dispensing device have been sterilized.

This methodology is decidedly costly and involves relatively long assembly times. Bearing in mind that the number of vials to be assembled to corresponding portable dispensing devices every day may reach numbers in the thousands of units, it is understood how simplifying and reducing or even limiting the time and costs for the sterilization and assembly steps for a single unit entail considerable economic advantages in terms of the final costs and production capacity.

As shown, the solutions of the prior art up to now are not able to guarantee a significant reduction in these times and costs.

### DISCLOSURE OF THE INVENTION

There is therefore a need to resolve the disadvantages and limitations mentioned with reference to the prior art.

This need is satisfied by an apparatus for sterilizing and assembling a cartridge containing a medical substance to a device for dispensing said medical substance according to claim 1 and by a method for sterilizing and assembling a cartridge containing a medical substance to a device for dispensing said medical substance according to claim 22.

### DESCRIPTION OF THE FIGURES

Further features and advantages of this invention will become clearer from the following detailed description of preferred non-limiting embodiments thereof, in which:

FIG. 1 is a front schematic view of a cartridge containing a medical substance and a device for dispensing a medical substance, prior to the relevant sterilization and assembly steps, according to an embodiment of this invention;

FIGS. 2-3 are front schematic views of the successive steps of sterilizing and assembling the cartridge and the device for dispensing a medical substance as shown in FIG. 1;

FIG. 4 is a front schematic view of the step of dispensing the medical substance from the cartridge that has previously been assembled and sterilized on a device for dispensing a medical substance, according to an embodiment of this invention.

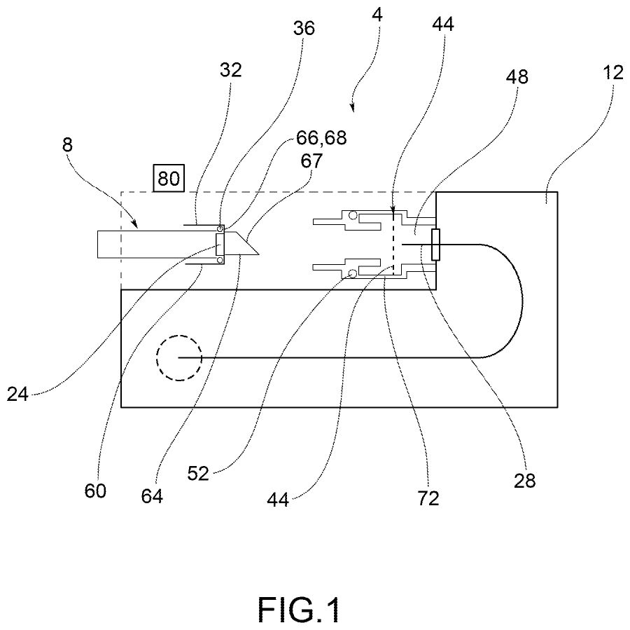

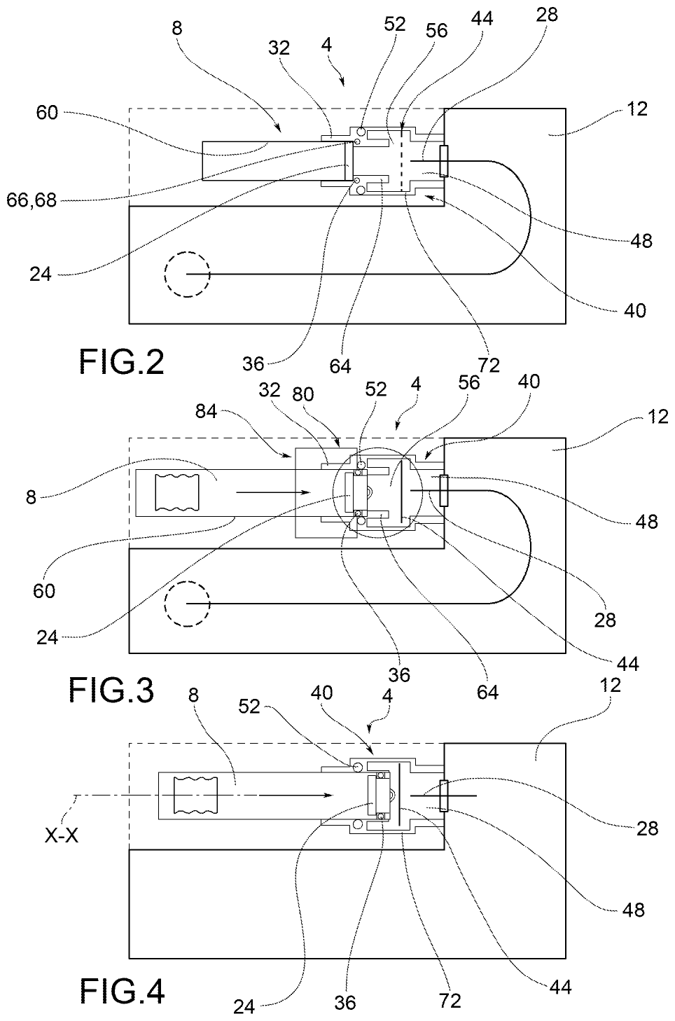

Elements or parts of elements common to the embodiments described in the following will be provided with the same reference signs.

### DETAILED DESCRIPTION

With reference to the aforementioned figures, reference sign 4 indicates, as a whole, an apparatus for sterilizing and assembling a cartridge 8 containing a medical substance to a device 12 for dispensing said medical substance by means of transdermal, intramuscular, subcutaneous or intravenous administration of said medical substance.

The dispensing device 12 may comprise a transdermal, intramuscular, subcutaneous or intravenous administration device 16 (known as a “POD”) which, in use, is applied to the skin of the patient or implanted into the body, and an activation device (known as a handheld device, not shown) for activating said administration device 16, which activation device is picked up by the user when required so as to activate the step of administering the medical substance, as is better described in the following.

More specifically, a dispensing device 12 according to this invention is a device that is suitable for performing medical treatments when placed in contact with a patient and when receiving energy from outside this dispensing device by temporarily positioning the dispensing device 12 in proximity to the POD when a medical treatment is required. It is also possible that the dispensing device is autonomous, i.e. has an internal energy supply and a microcontroller, and/or may be controlled by an external (handheld) device, for example by means of a wireless connection.

“In contact” means in cutaneous contact with the user, for example removably fixed to the skin of the patient, for example by means of an adhesive base, or more generally in contact with the body, including a device inside the body, fixed at least in part in a cavity of the body or implanted in the body.

According to one embodiment, the dispensing device 12 is an implanted device or a device partially inside the body and partially outside the body, for example a catheter, that is suitable for regulating the flow of a fluid. The dispensing device 12 may therefore be designed as a valve device suitable for enabling/disabling the flow of fluid or varying the flow rate of a fluid, or as a continuous dispensing device adapted to dispense a continuous flow of a medicine at a variable flow rate for a prolonged period of time. The medical treatment may therefore result from regulating the flow of a fluid from the outside into the body.

According to one possible embodiment, the dispensing device 12 is a device that is in contact with the skin by means of an adhesive or by means of a belt system that fixes it to the body. This device has a needle which preferably remains inserted in the body or is inserted and retracted each time it is necessary to dispense a dose of the medical substance.

According to one embodiment, the device may comprise a number of parts that are assembled together before use. The parts may be reusable parts and disposable parts.

With reference to this invention, the insertion of the cartridge into the dispensing device is to be understood in the same way if the cartridge is inserted into one of the parts that make up this dispensing device.

The sterilization and assembly apparatus 4 comprises a cartridge 8 containing a medical substance to be dispensed, the cartridge being provided with a cap 24. The cartridge 8 is pre-filled with the medical substance before being joined to the dispensing device 12. The cartridge 8 is preferably cylindrical and made of glass, but may also be made of a plastics material and have geometries other than cylindrical. Moreover, the cartridge 8 may be made of a flexible material. The cap 24 is usually made of rubber. The cap 24 may be fixed to the cartridge 8 by means of suitable supports which may be made of a plastics material or metal.

The device 12 for dispensing said medical substance is provided with at least one hollow needle 28 configured to pierce the cap 24 of the cartridge 8 and allow the withdrawal of the medical substance from the cartridge 8, in order to dispense said substance.

For the purposes of this invention, the specific typology of the dispensing device 12 used is not relevant; in other words, the means which allow the medical liquid to be pumped and suitably withdrawn from the cartridge 8 by means of the needle 28 may be of any kind.

The sterilization and assembly apparatus 4 advantageously comprises a first interface 32 that may be sealingly joined to the cap 24 of the cartridge 8 or to the cartridge itself by means of the interposition of a first gasket 36. The first gasket 36 is typically but not exclusively an O-ring.

The first interface 32, as better described in the following, is configured to be fitted or joined to the cartridge 8, and to create a mechanical connection to the dispensing device 12. Typically but not necessarily, the first interface 32 is made of a plastics material or a metal material or a rubber material.

The sterilization and assembly apparatus 4 comprises a second interface 40 joined to the dispensing device 12, near said needle 28. According to one possible embodiment, the second interface 40 is configured so as to couple to said first interface 32 and produce a mechanical connection between the cartridge 8 and the dispensing device 12. The coupling between the first interface 32 and the cartridge 8 is preferably a form-fitting coupling. In this embodiment, the first interface 32 and the second interface 40 are produced separately from one another and are then mechanically connected to one another, for example by means of a form-fitting coupling.

According to a further embodiment, the first interface 32 and the second interface 40 are in one piece so as to produce a single interface means that is fixed to the dispensing device 12 and is suitable for sealingly housing the cartridge 8 and/or the cap 24 of said cartridge 8.

The second interface 40 has a seal 44 that isolates the needle 28 from the external environment in a sterile volume 48. In other words, the needle 28 is enclosed within said delimited sterile volume 48, inside the second interface 40, by said seal 44. According to one possible embodiment, said seal 44 is a front seal 44 directly facing the first interface 32.

The second interface 40 is also provided with a second gasket 52 which may be sealingly joined to the first interface 32 in order to identify a sealed chamber 56 delimited between a portion of the cap 24 facing said needle 28 and said seal 44. The second gasket 52 is typically but not exclusively an O-ring. It should be noted that the sealed chamber 56 is understood to mean a sealed sterile chamber. Said sealed chamber 56 may also be provided with a unidirectional ventilation opening (not shown) that is suitable for preventing the pressure in the chamber from increasing when the cartridge 8 approaches the needle 28.

The second gasket 52 is preferably external to said sterile volume 48. The seal 44 is preferably but not exclusively a film, for example made of Tyvek®. It is also possible to use any other device that, in a first step, is suitable for preserving the sterility of said volume 48 and, in a second step, is suitable for being pierced in order to allow fluidic communication with said sealed chamber 56.

The first interface 32 preferably has a cylindrical, axial-symmetric configuration with respect to a main extension axis X-X.

According to one possible embodiment, the first interface 32 comprises a collar 60 that is counter-shaped with respect to the cap 24, and a first beaker 64 suitable for interfacing with the second interface 40, wherein the first beaker 64 has a narrowed section 66 with respect to the collar 60, so as to form a seat for said first gasket 36.

According to one possible embodiment, the first beaker 64 is configured to break the seal 44 of the second interface 40 when pressed against it.

For this purpose, according to one possible embodiment, the first beaker 64 has an open front end 67 provided with a beveled profile which is shaped so as to facilitate the piercing of the seal 44 when the cartridge 8, together with the relative first interface 32, is moved towards the second interface 40 provided with the seal 44.

According to one possible embodiment, the sterilization and assembly apparatus 4 comprises an end stop 68 for the movement of the cartridge 8 joined to the first interface 32 towards the needle 28. In particular, said end stop 68 is configured and positioned so as to stop the movement of the cartridge 8 before the first beaker 64 makes contact against the seal 44, wherein at the end stop 68 there is hermetic contact between the first beaker 64 and the second gasket 52 or between the cap 24 and the second gasket 52.

In particular, said end stop 68 stops the movement of the cartridge 8 towards the needle 28 before the latter pierces the cap 24 and the seal 44.

According to one possible embodiment, the end stop 68 is configured so as to allow an additional advance stroke of the cartridge 8 towards the needle 28, so as to allow the penetration of the needle 28 into the cap 24.

According to one possible embodiment, the second interface 40 comprises a cylindrical widened section 72 that forms a seat for said second gasket 52.

For example, said cylindrical widened section 72 is configured to form an end stop of the cartridge 8 at the narrowed section 66 of the first interface before the needle 28 has pierced the cap 24.

It should be noted that the end stop may be obtained both mechanically and by means of a suitable movement of the cartridge 8 and/or the needle 28. In other words, it is possible to use movement means (not shown) configured to translate the cartridge 8 and/or the needle 28 relative to one another along a direction parallel to said main extension axis X-X.

For example, said means for moving the cartridge 8 are configured to control the withdrawal and/or the extraction of the needle in order to allow said cap 24 to be pierced.

The sterilization and assembly apparatus 4 according to this invention also comprises a sterilizing device 80 configured to irradiate and sterilize said sealed chamber 56.

The sterilizing device 80 is configured to emit UV radiation and/or any type of irradiation known from the prior art for sterilization, for example an electron beam.

Said sterilizing device 80 is preferably configured to predominantly irradiate and sterilize said sealed chamber 56.

Said sterilizing device 80 is preferably completely external to and separate from the cartridge 8 and from the dispensing device 12; in other words, the sterilizing device 80 is part of the sterilization and assembly apparatus 4 but is completely independent and external to the cartridge 8 and the dispensing device 12.

According to one possible embodiment of this invention, in order to preserve the contents of the cartridge 8, the sterilization and assembly apparatus 4 comprises a mask 84 suitable for being fitted around the cartridge 8 near the cap 24, said mask 84 being impermeable or absorbent to the radiation used for sterilization, which radiation is emitted by the sterilizing device 80, so as to protect the medical substance contained in said cartridge 8. The mask 84 may also be suitable for covering specific regions of the dispensing device 12 or the cartridge so as to prevent the relative components from degrading as a result of the irradiation for sterilization.

According to one possible embodiment of this invention, said first interface 32 and said second interface 40 are configured so as to be at least partially permeable to the radiation for sterilization that is emitted by the sterilizing device 80, so as to penetrate into the chamber 56 in order to sterilize the inside thereof.

Said first interface 32 and said second interface 40 are preferably configured in such a way as to realize a mechanical coupling or mutual engagement, so as to mechanically fix the cartridge 8 to the dispensing device 12, near the needle 28 of the latter. Alternatively, the fixing is obtained by a coupling between the interface 40 and the cartridge itself.

The method according to this invention will now be described.

In particular, distinction should be made between the step of pre-assembling the dispensing device 12, in which the cartridge 8 is pre-assembled to this dispensing device 12, and the step of dispensing the medical substance.

The pre-assembly step takes place at the manufacturer of the dispensing device 12, while the step of dispensing the medical substance takes place directly when the device is used by the patient or the end user.

In particular, the method for sterilizing and assembling a cartridge 8 containing a medical substance to a device 12 for dispensing said medical substance comprises the steps of:
providing a cartridge 8 containing a medical substance to be dispensed, the cartridge 8 being provided with a cap 24,
providing a device 12 for dispensing said medical substance, which device is provided with at least one needle 28 configured to pierce the cap 24 of the cartridge 8 and allow the withdrawal of the medical substance from the cartridge 8,
providing a first interface 32 and sealingly joining it to the cap 24 of the cartridge 8, by means of the interposition of a first gasket 36,
providing a second interface 40 provided with a second gasket 52 and sealingly joining it to the dispensing device 12 near said needle 28, the second interface 40 being provided with a seal 44 that isolates the needle 28 from the external environment inside a sterile volume 48, the second gasket 52 being external to said sterile volume 48,
sealingly joining the first interface 32 to the second interface 40 in order to create a sealed chamber 56 between the seal 44 and the first interface 32, said sealed chamber 56 housing a portion of the cap 24 facing said needle 28,
providing a sterilizing device 80 configured to irradiate and sterilize said sealed chamber 56.

The step of sterilizing the sealed chamber 56 preferably takes place using a sterilizing device 80 that is external to and mechanically separate from the cartridge 8 and from the dispensing device 12.

According to one possible embodiment, the method comprises the step of providing a mask 84 suitable for being fitted around the cartridge 8 and/or near the cap 24, said mask 84 being impermeable or absorbent to the UV radiation or other radiation (for example an electron beam) emitted by the sterilizing device 80 so as to protect the medical substance contained in said cartridge 8 during the step of sterilizing the sealed chamber 56. As already stated, the mask 84 may be extended at least in part to the dispensing device 12 so as to prevent the components of the dispensing device or the material of the cartridge itself from being damaged by said irradiation for sterilization.

According to one possible embodiment, the method comprises the step of moving the cartridge 8 and the needle 24 towards one another so as to pierce the seal 44 with the first interface 32 after having sterilized the sealed chamber 56, without letting the needle 28 penetrate the cap 24. The final assembly and subsequent insertion of the portable device 12 into the “primary packaging” then takes place. It should be noted that, after moving the cartridge 8 and the dispensing device 12 towards one another, the step of piercing or breaking the seal 44 reduces the overall length of the dispensing device 12, which is therefore more compact.

The steps described above relate to the so-called “primary packaging,” i.e. they are carried out by the manufacturer of the dispensing device 12 which is then sold with the cartridge 8 pre-assembled to the dispensing device 12, and with the seal 44 already torn so as to place the previously sterilized volume 48 in communication with the sealed chamber 56 which was in turn sterilized by using the sterilizing device 80. It should be noted that the cartridge 8 is pre-filled with the medical substance before being joined to the dispensing device 12.

The subsequent step of dispensing the medical substance is carried out directly on the end user. For this purpose, the step of moving the cartridge 8 and the needle 28 towards one another so as to pierce the cap 24 with the needle 28 in a condition of use or dispensing the medical substance using the dispensing device 12 is provided, so as to obtain a fluidic connection which ends with the outlet needle for the medicine. It should be noted that the fluidic connection may be achieved by means of a single tube, tube components, or tube components and possible valves or pumps for regulating the flow of the liquid. The liquid may come out of the cartridge both by means of a syringe and by means of a pump which draws up the liquid.

In this way, the cap 24 is only pierced when the end user decides to administer the medical substance.

Before use, after having pierced the cap 24 with the needle 28, there may be a priming step, so as to allow any air contained in the cartridge 8 to escape, and/or a step of at least partially refilling the fluid part of the dispensing device 12.

As may be understood from the above, this invention overcomes the disadvantages of the prior art.

In particular, this invention makes it possible to sterilize and assemble the cartridge to the dispensing device extremely quickly and at reduced cost.

In fact, by virtue of this invention, it is possible to sterilize only the portions of the cartridge and the dispensing device that have to come into contact in order to be assembled together. In so doing, it is not necessary to sterilize the entire cartridge and/or the entire dispensing device, and it is not necessary to make the entire assembly area sterile, as is the case in the solutions from the prior art.

This results in a significant reduction in sterilization and assembly time and costs, by comparison with the solutions from the prior art.

Moreover, according to this invention, the manufacturer of the dispensing device may sterilize the sealed chamber delimited between the seal and the cap of the cartridge even before piercing said seal that delimits a sterile volume housing the needle with the fluid part.

The manufacturer of the dispensing device also breaks the seal so as to engage and mechanically connect the cartridge to the dispensing device as a result of the mechanical coupling obtained by means of the respective interfaces. The dispensing device is then provided to the end user in conditions in which the inside of the sealed chamber and the entire fluid part are sterile. Before the user administers the medical substance, the cap is pierced by the needle.

By virtue of the fact that the cartridge is pre-assembled to the dispensing device in a position close to the needle of the latter, it is possible to maintain an overall reduced dimension of the dispensing device which is sold to the end user already provided with the relevant cartridge.

Moreover, as described above, the dispensing device has an overall reduced length, by virtue of the fact that the cartridge is moved towards the needle of the dispensing device after the seal has been broken.

A person skilled in the art, in order to satisfy contingent and specific needs, may make numerous modifications and variations to the devices and methods described above, which modifications and variations are all contained within the scope of the invention as defined in the following claims.

1. An apparatus for sterilizing and assembling a cartridge containing a medical substance to a device for dispensing said medical substance, comprising:
a cartridge containing the medical substance to be dispensed, the cartridge being provided with a cap,
a device for dispensing said medical substance, which device is provided with at least one hollow needle configured to pierce the cap of the cartridge and allow the withdrawal of the medical substance from the cartridge, in order to dispense said substance,
a first interface that may be sealingly joined to the cap or to a portion of the cartridge,
a second interface joined to the dispensing device near said needle,
wherein the second interface has a seal that isolates the needle from the external environment in a sterile volume, and is provided with a second gasket which may be sealingly joined to the first interface or to the cartridge itself in order to identify a sealed chamber delimited between a portion of the cap facing said needle and said seal,
a sterilizing device configured to irradiate and sterilize said sealed chamber.

2. The sterilization and assembly apparatus according to claim 1, wherein the first interface may be sealingly joined to the cap of the cartridge by means of the interposition of a first gasket, and wherein the second gasket is external to said sterile volume.

3. The sterilization and assembly apparatus according to claim 1, wherein the first interface has a cylindrical, axial-symmetric configuration with respect to a main extension axis.

4. The sterilization and assembly apparatus according to claim 1, wherein the first interface comprises a collar that is counter-shaped with respect to the cap.

5. The sterilization and assembly apparatus according to claim 4, wherein the first interface comprises a first beaker comprised in the sealed chamber and suitable for interfacing with the second interface, wherein the first beaker has a narrowed section with respect to the collar.

6. The sterilization and assembly apparatus according to claim 1, comprising, inside the sealed chamber, a first beaker configured to break the seal of the second interface when pressed against it.

7. The sterilization and assembly apparatus according to claim 1, wherein the first interface is configured to break the seal of the second interface when pressed against it.

8. The sterilization and assembly apparatus according to claim 1, wherein the seal is a film.

9. The sterilization and assembly apparatus according to claim 1, wherein the apparatus comprises an end stop for the movement of the cartridge provided with the first interface towards the needle, said end stop being configured and positioned so as to stop the movement of the cartridge before the first beaker makes contact against the seal, wherein at the end stop there is hermetic contact between the first beaker and the second gasket.

10. The sterilization and assembly apparatus according to claim 9, wherein said end stop stops the movement of the cartridge towards the needle before the latter pierces the cap and the seal.

11. The sterilization and assembly apparatus according to claim 9, wherein the end stop is configured so as to allow an additional advance stroke of the cartridge towards the needle, so as to allow the penetration of the needle into the cap.

12. The sterilization and assembly apparatus according to claim 1, wherein the second interface comprises a cylindrical widened section that forms a seat for said second gasket.

13. The sterilization and assembly apparatus according to claim 12, wherein said cylindrical widened section is configured to form an end stop of the cartridge at a narrowed section of the first interface after the needle has pierced the cap.

14. The sterilization and assembly apparatus according to claim 1, wherein the sterilizing device is configured to emit UV radiation and/or a sterilization beam.

15. The sterilization and assembly apparatus according to claim 1, wherein the sterilizing device is configured to irradiate and sterilize only said sealed chamber or around it.

16. The sterilization and assembly apparatus according to claim 1, comprising a mask suitable for being fitted around the cartridge near the cap or part of the cartridge or the device, said mask being impermeable to the radiation and/or the sterilizing beam emitted by the sterilizing device so as to protect the medical substance contained in said cartridge and/or the components of the dispensing device.

17. The sterilization and assembly apparatus according to claim 1, wherein said sterilizing device is completely external to and separate from the cartridge and from the dispensing device.

18. The sterilization and assembly apparatus according to claim 1, wherein the sterilization and assembly apparatus comprises means for moving the cartridge that are configured to move the cartridge and/or the needle towards one another, enabling the piercing of said seal and/or said cap.

19. The sterilization and assembly apparatus according to claim 1, wherein said first interface and said second interface are configured so as to be at least partially permeable to the radiation and/or to the electrons emitted by the sterilizing device.

20. The sterilization and assembly apparatus according to claim 1, wherein said first interface and said second interface are configured in such a way as to realize a mechanical coupling or mutual engagement, so as to mechanically fix the cartridge to the dispensing device near the needle of the latter.

21. The sterilization and assembly apparatus according to claim 1, wherein the first interface and the second interface are in one piece so as to realize a single interface means that is fixed to the dispensing device and is suitable for sealingly housing the cartridge and/or the cap of said cartridge.

22. A method for sterilizing and assembling a cartridge containing a medical substance to a device for dispensing said medical substance, comprising the steps of:
providing a cartridge containing a medical substance to be dispensed, the cartridge being provided with a cap,
providing a device for dispensing said medical substance, which device is provided with at least one needle configured to pierce the cap of the cartridge and allow the withdrawal of the medical substance from the cartridge,
providing a first interface and sealingly joining it to the cap of the cartridge by means of the interposition of a first gasket,
providing a second interface provided with a second gasket and sealingly joining it to the dispensing device near said needle, the second interface being provided with a seal that isolates the needle from the external environment inside a sterile volume, the second gasket being external to said sterile volume,
sealingly joining the first interface to the second interface in order to create a sealed chamber between the seal and the first interface, said sealed chamber housing a portion of the cap facing said needle,
providing a sterilizing device configured to irradiate and sterilize said sealed chamber.

23. The sterilization and assembly method according to claim 22, comprising the step of moving the cartridge and the needle towards one another so as to pierce the seal with the first interface after having sterilized the sealed chamber, without letting the needle penetrate the cap, in the condition of non-use of the dispensing device or ‘primary packaging’.

24. The sterilization and assembly method according to claim 23, comprising the step of moving the cartridge and the needle towards one another so as to pierce the cap with the needle in the condition of use or dispensing the medical substance using the dispensing device.

25. The sterilization and assembly method according to claim 22, comprising the step of providing a mask suitable for being fitted around the cartridge near the cap, said mask being impermeable to the radiation and/or to the electron beam emitted by the sterilizing device so as to protect the medical substance contained in said cartridge during the step of sterilizing the sealed chamber.

26. The sterilization and assembly method according to claim 22, wherein the step of sterilizing the sealed chamber is carried out by means of said sterilizing device external to and separate from the cartridge and also from the dispensing device.

27. The sterilization and assembly method according to claim 22, wherein the first interface and the second interface are in one piece so as to realize a single interface means that is fixed to the dispensing device and sealingly houses the cartridge and/or the cap of said cartridge.
